(19) United States
(12) Patent Application Publication
(10) Pub. No.: US 2021/0383916 A1
(43) Pub. Date: Dec. 9, 2021

(54) INTELLIGENT RADIATION DOSE MONITORING

(71) Applicant: CERNER INNOVATION, INC., Kansas City, KS (US)

(72) Inventors: Satya Krishna Pillarisetty, Bangalore (IN); Huong Vu, Overland Park, KS (US)

(21) Appl. No.: 16/894,124

(22) Filed: Jun. 5, 2020

(51) Int. Cl. G16H 40/20; G16H 40/63; G16H 10/60; G16H 50/20; A61B 6/00; G01T 1/02; G01T 7/12

(52) U.S. Cl. CPC G16H 40/20; G16H 40/63; G16H 10/60; G01T 7/125; A61B 6/542; G01T 1/02; G16H 50/20

(57) ABSTRACT

Embodiments herein disclose systems, methods, and computer-readable media for intelligently monitoring radiation exposure values. The intelligent monitoring can monitor an amount of radiation a clinician is exposed to (via exams already performed) and identify how much radiation is acceptable for continued exposure before exceeding an exposure threshold value within a predetermined time period. Additionally, the system described herein can provide insights into how many procedures of a particular type a clinician is available to complete before expiration of the predetermined time period. The monitoring data can be provided via a graphical user interface and can be integrated into a scheduling system, an electronic health records system, etc., to provide meaningful insights for resource management.

### BACKGROUND

The use of radiological procedures is ever increasing in today's healthcare environment. In turn, clinicians are subjected to increased exposure to ionizing radiation. While a patient's exposure to ionizing radiation has been a subject of interest, a clinician's exposure and monitoring thereof has, thus far, been neglected. Failure to monitor a clinician's exposure to ionizing radiation can lead to overexposure. Overexposure to ionizing radiation can lead to several health problems such as cancer, cardiovascular disease, and the like.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description and Drawings.

In brief and at a high level, embodiments of the present invention provide systems, methods, and computer-readable media for intelligent radiation dose monitoring. Embodiments provide an application that coordinates with various components of a system to intelligently monitor radiation dose exposure and facilitate effective resource management.

One embodiment provides one or more non-transitory computer-readable media having computer-executable instructions embodied thereon that, when executed by a processor of a computer device, perform a method. In accordance with the media, the method comprises receiving an indication to schedule a first clinician for a first exam; identifying an estimated exposure associated with the first exam, wherein the estimated exposure for the first exam is based on one or more of a historical dose associated with the first exam and a historical dose associated with the first clinician for the first exam; identifying a remaining exposure value for the first clinician, wherein the remaining exposure value is an amount of radiation exposure permitted prior to meeting or exceeding an exposure threshold value; determining that the remaining exposure value for the first clinician is less than the estimated exposure associated with the first exam; and generating an alert that the first clinician will exceed the exposure threshold value if scheduled to perform the first exam.

Another embodiment provides one or more non-transitory computer-readable media having computer-executable instructions embodied thereon that, when executed by a processor of a computer device, perform a method. In accordance with the media, the method comprises identifying a request to generate an exposure tracking graphical user interface (GUI) for a first clinician for a first exam; identifying whether a dosimeter for the first clinician is operational to access a dosimeter exposure value identified for the first exam; upon determining that the dosimeter is not operational, providing an alert to the first clinician that the dosimeter is not operational; identifying a variable dose value for the first exam, wherein the variable dose value is identified based on one or more of an output of the radiation system used for the first exam and an exposure value associated with one or more other personnel present during the first exam; identifying a net dose value, wherein the net dose value is a sum of the dosimeter value and the variable dose value; indicating a remaining exposure value based on an exposure threshold value and the net dose value; and generating the GUI including at least an indication of the remaining exposure value and at least one indication of a second exam the first clinician is eligible to perform based on the remaining exposure value.

Yet another embodiment provides a system for intelligent radiation dose exposure monitoring. The system comprises one or more processors configured to: receive an indication to schedule a first clinician for a first exam; identify an estimated exposure associated with the first exam, wherein the estimated exposure for the first exam is based on one or more of a historical dose associated with the first exam and a historical dose associated with the first clinician for the first exam; identify a remaining exposure value for the first clinician, wherein the remaining exposure value is an amount of radiation exposure permitted prior to meeting or exceeding an exposure threshold value; determine that the remaining exposure value for the first clinician is less than the estimated exposure associated with the first exam; and generate an alert that the first clinician will exceed the exposure threshold value if scheduled to perform the first exam.

### BRIEF DESCRIPTION OF THE DRAWINGS

Embodiments are described in detail below with reference to the attached drawings figures, wherein:

FIG. 1 depicts a block diagram of an exemplary system architecture in accordance with an embodiment of the present invention;

FIG. 2 is a flow diagram of an exemplary method in accordance with an embodiment of the present invention;

FIG. 3 is depicts an exemplary user interface in accordance with an embodiment of the present invention;

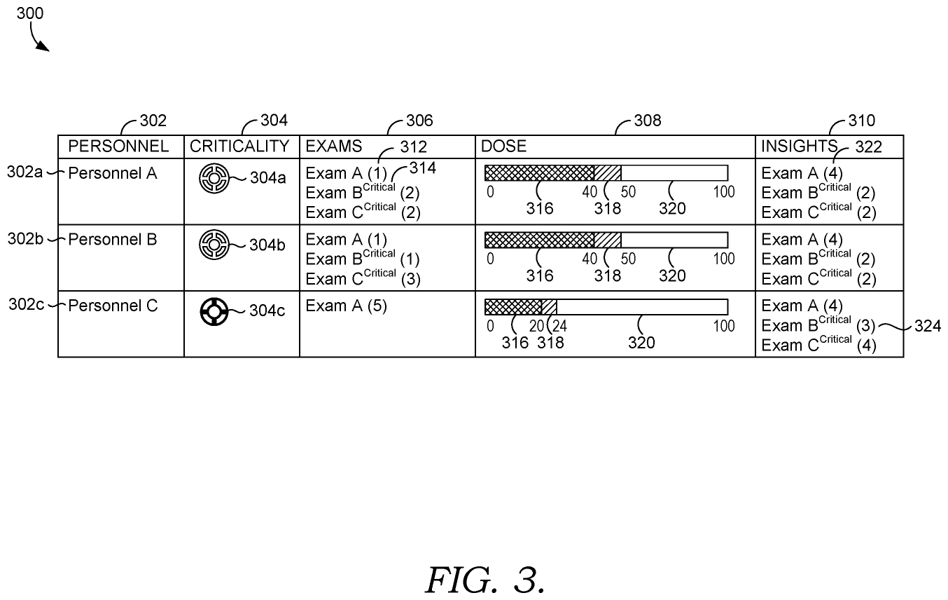

FIG. 4 is a flow diagram of an exemplary method in accordance with an embodiment of the present invention;

FIG. 5 is a flow diagram of an exemplary method in accordance with an embodiment of the present invention; and

FIG. 6 depicts a block diagram of an exemplary computing environment suitable to implement embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of the disclosure may be embodied as, among other things: a method, system, or a set of instructions embodied on one or more computer-readable media. Accordingly, the embodiments may take the form of a hardware embodiment, a software embodiment, or an embodiment combining software and hardware. In one embodiment, the invention takes the form of a computer-program product that includes computer-useable instructions embodied on one or more computer-readable media, as discussed further herein.

Embodiments of the present invention provide systems, methods, and computer-readable media for intelligent radiation dose monitoring. At a high level, embodiments of the present invention provide a customized and complex software product that specifically addresses a need to integrate radiation dose monitoring technology with electronic health record (EHR) technology, resource management technology, and the like. The software product can communicate with one or more disparate sources to, among other things, provide radiation dose exposure data from a data store, store radiation dose exposure data within a variety of sources (e.g., a data store, a scheduling system, an EHR, etc.), generate one or more recommendations based on the radiation dose exposure, and the like.

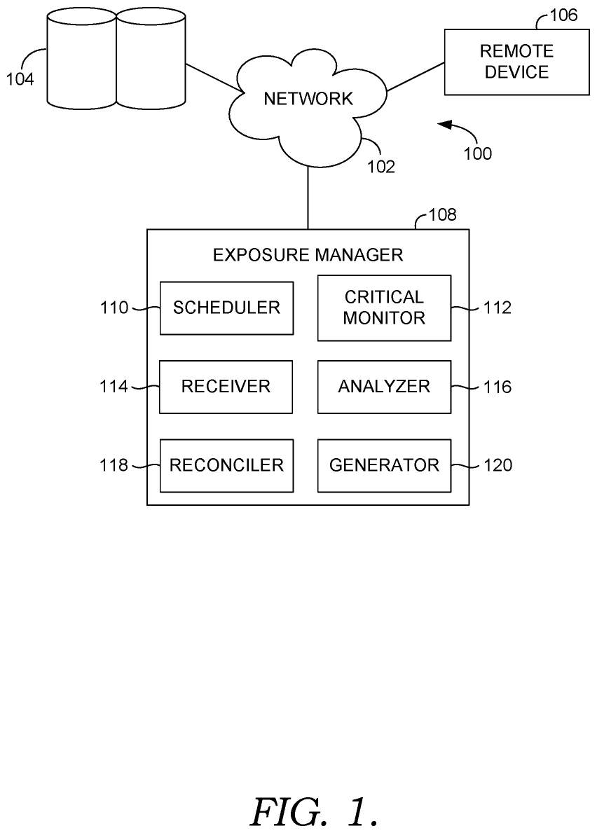

Referring to the drawings in general, an initially to FIG. 1, a block diagram illustrating an exemplary system 100 architecture in which some embodiments of the present disclosure may be employed. It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory.

It should be understood that the system 100 shown in FIG. 1 is an example of one suitable computing system architecture. Each of the components of FIG. 1 may be implemented via any type of computing device. The components can communicate with each other via a network including, without limitation, one or more local area networks (LANs) and/or wide area networks (WANs). Such networking environments are commonplace in offices, enterprise-wide computer networks, intranets, and the Internet. It should be understood that any number of components shown in FIG. 1 may be employed within the system 100 within the scope of the present invention. Each may be implemented via a single device or multiple devices cooperating in a distributed environment. Additionally, other components not shown may also be included within the environment.

Among other components not shown, the system 100 includes a variety of user devices, such as remote device 106, a network 102, a data store 104, and an exposure manager 108, any of which can interact with any other component of the system 100 and each of which are communicatively coupled with each other. These components may communicate with each other via networking means (e.g., network 102) which may include, without limitation, one or more local area networks LANs and/or wide area networks (WANs). In exemplary implementations, such networks comprise the Internet and/or cellular networks, amongst any of a variety of possible public and/or private networks.

Remote device 106 can comprise any type of computing device capable of use by a user. By way of example and not limitation, a remote device can be embodied as a personal computer (PC), a laptop computer, a mobile device, a smartphone, a tablet computer, a smart watch, a wearable computer, a fitness tracker, a personal digital assistant (PDA) device, a global positioning system (GPS) device, a video player, a handheld communications device, an embedded system controller, a camera, a remote control, a wearable electronic device with a camera (e.g., smart glasses, gesture-based wearable computers, etc.) a consumer electronic device, a workstation, or any combination of these delineated devices, a combination of these devices, or any other suitable computer device.

Data store 104 can store a variety of data. While shown as a single database in FIG. 1, data store 104 can be multiple data stores and can be associated with different entities. For the sake of simplicity, data store 110 is described herein as a singular database that is in communication with multiple entities. One of skill in the art will understand the data store 104 can take a variety of forms, be represented as multiple components, and communicate with any number of sources. Data store 104 can include, among other things, health-related data for individuals such as EHRs, radiology data (discussed further herein) such as procedures performed and radiation dose exposure, patient identifiers, demographic information, and the like.

Exposure manager 108 comprises instructions to perform intelligent monitoring of radiation dose exposure. In particular, and as described further herein, exposure manager 108 can receive information from a variety of data sources to monitor radiation dose exposure for clinicians, patients, and the like.

Exposure manager 108 can facilitate communication between a remote device, such as remote device 106, and a plurality of other sources such as an EHR system, a data store (such as data store 104), and the like. Exposure manager 108 can include an application programming interface (API) library that includes specifications for routines, data structures, object classes, and variables that support the interaction of the exposure manager 108 architecture and the software framework of one or more disparate sources (e.g., an EHR server). These APIs can include configuration specifications for the system 100 such that the components therein may communicate with each other, as described herein.

Exposure manager 108 comprises scheduler 110, critical monitor 112, receiver 114, analyzer 116, reconciler 118, and generator 120. In the present invention, radiation dose monitoring is achieved by integration of a plurality of sources with exposure manager 108. Initially, radiation dose exposure is obtained from one or more sources. A common source is a dosimeter. A dosimeter, as used herein, is a device used to measure an absorbed dose of ionizing radiation. Clinicians are currently instructed to wear dosimeters but there is no tracking of net dose exposure of clinicians and the information is not tied to scheduling/resource utilization. Net dose exposure cannot be reflected by dosimeters due to non-adherence of malfunctioning of devices. Thus, variable dose values can be utilized herein, as described below.

Exposure manager 108 comprises scheduler 110 that is integrated with a scheduling application (not shown) utilized to schedule exams/procedures; in particular, a scheduling application utilized to schedule radiological exams (e.g., x-rays, etc.). Note that herein the term “exam” is used to refer to any activity where ionizing radiation is emitted. Scheduler 110 can receive indications of an exam to be scheduled for a patient, a clinician to schedule for the exam, and the like. For instance, a scheduling personnel may initiate a scheduling application that interfaces with exposure manager 108. Scheduler 110 can identify an exam that is to be scheduled for a patient or a clinician that a scheduling personnel is trying to assign to an exam to be scheduled or an exam that is already scheduled to occur.

In the aspect of scheduling, exposure manager 108 comprises critical monitor 112. Exams can be associated with an exam critical indicator, where an exam critical indicator of “critical” indicates an exam that is “critical” and an exam critical indicator of “non-critical” indicates an exam that is “non-critical.” As used herein, a critical exam refers generally to an exam that is associated with a predetermined number of criteria, as designed by a user (i.e., a user that has been given editable controls related to criteria creation and the like). For instance, a critical exam can refer to an exam with a mortality rate of a specified value (e.g., over 50%). In another example, a critical exam can refer to an exam that requires a clinician of a certain specialty (e.g., cardiologists are required for exams pertaining to the heart).

Further, clinicians can also be associated with a clinician critical indicator. A clinician critical indicator of “critical” indicates that a clinician is “critical” while a clinician critical indicator of “non-critical” indicates that the clinician is non-critical. A critical clinician, as used herein, refers generally to a clinician that should be reserved for critical exams. This reservation can be based on expertise (e.g., a clinician has performed over a threshold number of a particular type of exam), success rate (e.g., a clinician has a very low mortality rate for a particular type of exam), specialty (e.g., a clinician holds a certification/specialty that is required for a particular type of exam), and the like. A non-critical clinician, as used herein, is a clinician that either does not have to be reserved for critical exams (but may perform them) or cannot perform critical exams (e.g., certification required for a critical exam is not held by the non-critical clinician). Clinician critical indicators can be configurable by a user that has, as above, been given editable controls related to clinician critical indicators.

Radiation dose exposure data can be collected related to particular exams, clinicians, patients, and the like. In particular, Radiological exam A can be associated with an exposure of X on average. However, Radiological exam A can be associated with an exposure of X for Clinician A but an exposure of Y for Clinician B. As previously mentioned, radiation dose exposure can be collected from dosimeters by, for instance, receiver 114. Receiver 114 can receive dose exposure data from one or more devices and/or systems. Dosimeters can be devices that interface with receiver 114 to communicate dosimeter exposure values. There are instances where dosimeter exposure values may not be available. For example, a dosimeter could be non-operational (i.e., not functioning) for any number of reasons (e.g., low battery, device defect, etc.) or a clinician could have neglected to obtain a dosimeter prior to an exam. In those situations where dosimeter data is not available, other data sources can be evaluated to identify a radiation dose for an exam in question. For instance, an amount of radiation emitted from the radiation system can be applied to the exposure for the clinician (e.g., the radiation system emit values can be accessible by receiver 114 and can be part of a disparate system). Alternatively, an amount of radiation associated with the patient can be applied to the exposure for the clinician. Further, if at least one other personnel member is present in the exam, the amount of radiation associated with the at least one other personnel (or an average if more than one other personnel member is present) can be applied to the exposure for the clinician. Radiation dose data acquired from any source other than a dosimeter is referred to herein as a variable dose value. Thus, receiver 114 can receive radiation dose exposure data from a dosimeter exposure value or variable dose values. A net dose value is the sum of the dosimeter exposure value and the variable dose value.

Additionally, receiver 114 can be integrated with an alerting capability such that alerts related to a non-functioning dosimeter are generated. For instance, a clinician could be alerted that their dosimeter is not functioning by way of an alert to another device associated with the clinician (e.g., a wearable, a mobile device, etc.) or the radiology system. Alerts can also be triggers upon starting the exam in an electronic health record. Sensors could also be utilized so that another clinician present during the exam could be notified of the non-functioning status as well. Further, any number of clinicians or systems could be alerted to an absence of a dosimeter for any clinician present in an exam.

Once the exposure values are received, either dosimeter exposure values, variable dose values, or a combination thereof, analyzer 116 evaluates the received data with respect to an exposure threshold value. Exposure threshold values, as used herein, are predetermined values (e.g., numerical values, percentages, etc.) that are configurable by an authorized user (i.e., a user that has been given editable controls related to exposure threshold values). Exposure threshold values can be applied for a specific period of time such as a daily threshold, weekly, monthly, etc. Several organizations have recommended thresholds including the American College of Radiology and the American College of Cardiology. It should be noted that any one of the configurable metrics described herein (e.g., exposure threshold values, clinician critical indicators, exam criticality, etc., can be configured by users as described herein or can be determined by SML (standard machine learning programming language).

Analyzer 116 can evaluate a net dose exposure value with respect to a remaining exposure value. A remaining exposure value, as used herein, refers to the exposure threshold value less the net dose exposure value.

Exposure Threshold Value−Net Dose Exposure Value=Remaining Exposure Value

Once the remaining exposure value is identified, it is associated with a clinician and can be utilized by scheduler 110 to schedule clinicians for future exams. In doing so, an estimate exposure is associated with the exam to be scheduled. An estimated exposure for an exam can be inferred from a historical dose associated with the exam to be scheduled. This can be an average value based on the radiation doses associated with previous exams of the same type as the exam to be scheduled. Additionally, an estimated exposure for an exam can be identified on a per-clinician basis. For instance, an estimated exposure for Exam A may be value X but the estimated exposure for Exam A with Clinician B is value Y (e.g., Clinician B may be faster than Clinician Z at performing Exam A so the estimated exposure for Exam A may be lower for Clinician B than for Clinician Z). This information can be identified by analyzing historical exam data for clinicians. The exam data can be from a variety of disparate sources/systems such that the estimated exposures can be per-clinician, per-site, regional values, and the like. The exam data can be from imported exam data from one or more disparate sources. For example, the exam data can be previous exams performed by a clinician at a different facility (e.g., a south location of a hospital vs a north location of the same hospital). This imported data can be mapped against the exam data in the current system to provide a cumulative view of exposure for an individual.

In situations where a remaining exposure value for a particular clinician is greater than or equal to an estimated exposure value of an exam, that clinician can be selected for the exam as the exam will not result in exceeding the exposure threshold value. If, however, a remaining exposure value for a particular clinician is less than the estimated exposure value of an exam, that clinician cannot be selected for the exam. In those situations, reconciler 118 can propose alternative clinician options when the initial clinician evaluated will not work (i.e., when the initial clinician does not have enough remaining exposure to absorb the estimated exposure value of the exam).

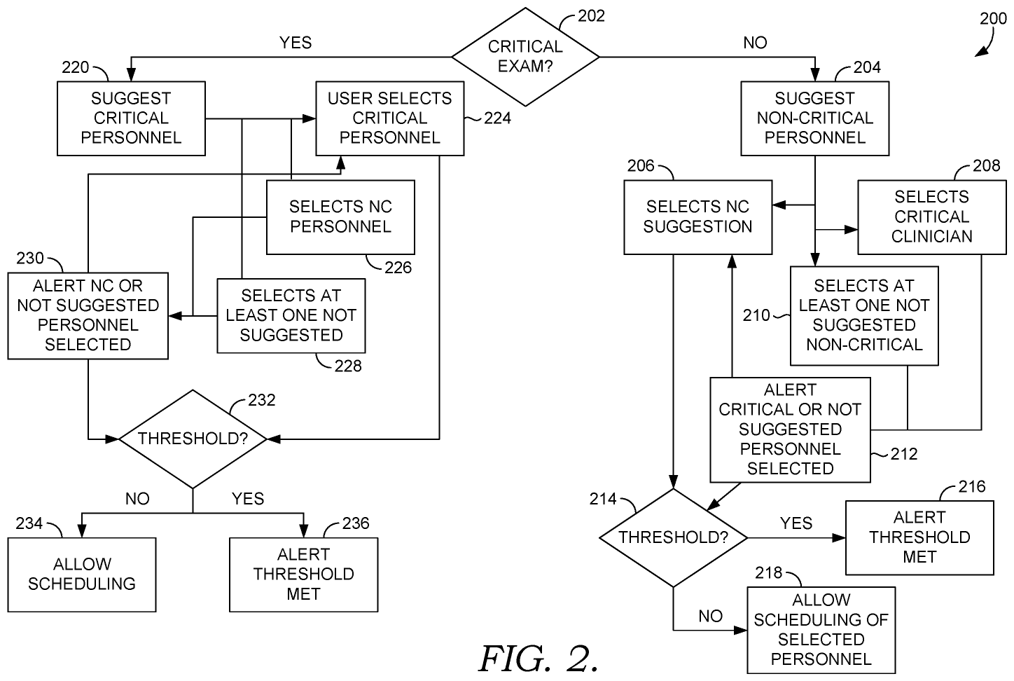

The reconciler 118 can initially identify, based on the clinician critical indicator, the exam critical indication, or a combination thereof, an initial pool of alternate clinicians to suggest. This decision flow is illustrated in FIG. 2 with flow 200. Initially, a decision is made whether the exam is associated with an exam critical indicator of “critical” or “non-critical” at block 202. Put simply, if the exam to be scheduled is critical, a clinician associated with a clinician critical indicator of “critical” is required and any clinician associated with a “non-critical” status would be filtered out. Conversely, any exam to be scheduled that is non-critical could be performed by clinicians associated with either a “critical” or “non-critical” status. In an embodiment, “non-critical” exams are satisfied with “non-critical” clinician suggestions. Thus, based on determining at block 202 that the exam is not critical, one or more personnel associated with a “non-critical” clinician critical indicator is suggested at block 204. In embodiments, only the “non-critical” suggested clinicians are available for selection. In other embodiments, the “non-critical” suggested clinicians are provided along with the other clinicians but the suggested clinicians are distinguished from the other clinicians (e.g., the suggested clinicians can be highlighted, associated with a suggested/preferred indicator, or the like). Any known method for distinguishing options can be used.

Based on the suggestion, a user has three possible outcomes in the event that all clinicians are still displayed. A user can selected one of the non-critical suggested clinicians at block 206, the user can selected a critical clinician at block 208, or the user can select at least one non-critical clinician that was not suggested at block 210. In the event the user selects a critical clinician at block 208 or at least one non-critical clinician that was not suggested at block 210, an alert is generated at block 212 that a critical clinician or a clinician that was not suggested was selected. The alert generated at block 212 can be overridden such that the method proceeds to block 214 to determine if the selected clinician will meet the threshold if scheduled for the exam or a user could select one of the non-critical clinicians originally suggested at block 206, which also results in proceeding to block 214. At block 214, a determination is made as to whether or not the selected clinician will exceed their threshold by performing the exam. This determination utilizes information compiled by the receiver 114 such as dosimeter exposure values, variable does values, remaining exposure values, and the like. In short, the threshold determination relies on a clinician's remaining exposure value being greater than an estimated exposure for the exam in questions.

If the threshold is not exceeded, scheduling of the selected personnel is permitted at block 218. If the threshold will be met or exceeded, an alert is generated at block 216 that the threshold is met and scheduling of the selected personnel should not proceed. Suggested clinicians can also be provided with the alert at block 216. This process can be performed by the exposure manager 108 and components thereof such as the critical monitor 112 to determine critical indicators (for exams and clinicians), the analyzer 116 to determine if the threshold is met, the reconciler 118 to provide suggested clinicians, and the like.

Returning back to decision block 202, if the exam is determined to be critical, the system suggests one or more critical personnel to select for the exam at block 220. At this point, a user again has three potential paths forward: the user can select one of the suggested critical personnel at block 224, the user can select a non-critical personnel at block 226, or the user can select at least one clinician that was not suggested at block 228. In the event that the user makes a selection at block 226 or 228, an alert is generated at block 230 that indicates a non-critical clinician or a clinician that was not suggested at block 220 was selected. From there, a user can return to block 224 and select a suggested critical personnel or the user can override the alert and proceed to decision block 232. When a user selects a critical personnel at block 224, the method also proceeds to decision block 232. At block 232, a decision is made whether the threshold is met or exceeded for the selected clinician by performance of the exam. If the threshold is not met, scheduling of the selected clinician is permitted at block 234. If the threshold is met or exceeded, an alert is generated at block 236 that scheduling of the selected clinician should not occur. One or more suggested clinicians can be provided with the alert.

In embodiments, the suggested personnel (i.e., the suggested clinicians at block 220 and 204) are all clinicians with a clinician critical indicator that match the exam critical indicator. In other words, for a critical exam, all critical clinicians are suggested and vice versa. In additional embodiments, the suggested personnel in block 220 and 204 are already filtered such that only clinicians that are associated with a clinician critical indicator that match the exam and have a remaining exposure value that is greater than or equal to the estimated exposure of the exam are suggested. In that event, the threshold determinations of block 214 and 232 can be applied to all clinicians prior to the suggestion.

Returning to FIG. 1, generator 120 can generate a graphical user interface (GUI) 300 to provide the above-described information. The GUI 300 can comprise, among other things, a personnel area 302, a criticality area 304, an exams performed area 306, a dose area 308, and an insights area 310. The personnel area 302 can include one or more personnel illustrated by Personnel 302a-302c. The criticality area 304 can include a clinician critical indicator for each personnel listed in personnel area 302. For example, the critical indicator 304a and 304b for Personnel A and Personnel B are shown as critical while the critical indicator 304c for Personnel C is shown as non-critical. The critical indicators can be depicted in any known fashion to distinguish between critical and non-critical.

The exams performed area 306 includes a listing of one or more exams already-performed by the associated personnel and a numerical value for the number of times the exam has been performed. For instance, Personnel A has already completed Exam A one-time and both Exams B and C twice. Note that Exams B and C are critical exams by inclusion of an exam critical indicator 314. Personnel C, as a non-critical clinician, has not performed any critical exams.

The dose area 308 includes a graphical representation of radiation dose exposure for the respective clinician. The graphical representation is shown in GUI 300 as a bar graph but any graphical depiction could be utilized. Further, a textual depiction could also be used to represent the radiation dose exposure. The dose area, regardless of the medium of depiction, can include a representation of a dosimeter exposure value, a representation of a variable dose value, and a representation of a remaining exposure value. Conversely, the values themselves can be provided in conjunction with the representations or alone within the GUI 300. To illustrate, dose area 308 includes a representation for Personnel A that indicates a representation of a dosimeter value 316, a representation of a variable dose value 318, and a representation of a remaining exposure value 320 (also shown as representations 316, 318, and 320 for Personnel B and C). The representations are also accompanied by the numerical values. For instance, for Personnel A, the dosimeter exposure value is shown to be 40, while the variable dose value is 10. The variable dose value is surmised by the presence of a net dose value 50, which is the sum of the dosimeter exposure value and the variable dose value. An exposure threshold value of 100 is also represented. Personnel B has the same breakdown as Personnel A with respect to the net dose value, even while performing different exams. Personnel C, however, is shown to have a dosimeter exposure value of 20 and a variable dose value of 4, which results in a net dose value of 24. Thus, the representation of the remaining exposure value 320 for Personnel C is much larger than Personnel A and B.

The insights area 310 can include on or more exams to be completed with a numerical indication 322 of how many of said exam can be completed by the respective personnel. For instance, based on accessing historical exposure values for a clinician's previous exams, the system can predict how many of a particular exam a clinician is available to perform before meeting or exceeding their threshold. For example, Personnel A is predicted to be able to perform Exam A 4 more times. The insights area 310 can provide insights that are cumulative (i.e., the number of times a clinician is expected to perform a particular exam is cumulative with the other exams listed) or individually (i.e., the number of times a clinician is expected to perform a particular exam is based solely on that exam's exposure, not a culmination of exams performed by the clinician).

While the above has been described within the context of resource management and scheduling, there are many applications of the present invention beyond the scope of scheduling. For example, the above monitoring of clinician exposure is also a valuable tool for instances when a clinician becomes a patient. Additional embodiments allow the system to communicate with an EHR to import exposure values of clinicians to their EHR. For instance, the EHR of the clinician can display the exposure radiation dose of the clinician (when the clinician is a patient). This is valuable information when scheduling the clinician, now a patient, for radiological exams. Depending on the clinician/patient's exposure through their occupation, different treatment plans may need to be evaluated to minimize additional exposure when available. Additionally, as previously mentioned, the exposure values of the clinician can be imported from one or more disparate systems. For instance, a clinician can perform exams at a plurality of facilities such that exposure values from each facility are needed to get a cumulative view of the clinician's actual exposure. In another embodiment, a clinician could relocate such that past exposure values should be imported to gain a cumulative view of the previous exposure history for the clinician. Furthermore, an individual, be it a patient or a clinician-turned-patient, can be associated with an exposure value that travels with their EHR such that it is available regardless of location of treatment.

In additional embodiments, exposure values for a plurality of clinicians can be aggregated to identify a cumulative clinician exposure value. This could be at a specific location, for a specific facility, and the like. This can allow users (e.g., hospital administrators, third party vendors, etc.) to view a cumulative clinician exposure value in comparison with a goal exposure threshold for the group. This provides a tool for users to make changes to control an overall exposure for a population (e.g., cardiological radiologists can be a population of the plurality of clinicians).

To summarize the above, exposure manager 108 can receive and analyze data related to radiation dose exposure from a variety of sources. Exposure manager 108 can generate insights into a capacity of a clinician related to radiation dose exposure such that exposure thresholds are not exceeded and clinicians are utilized where they are needed most.

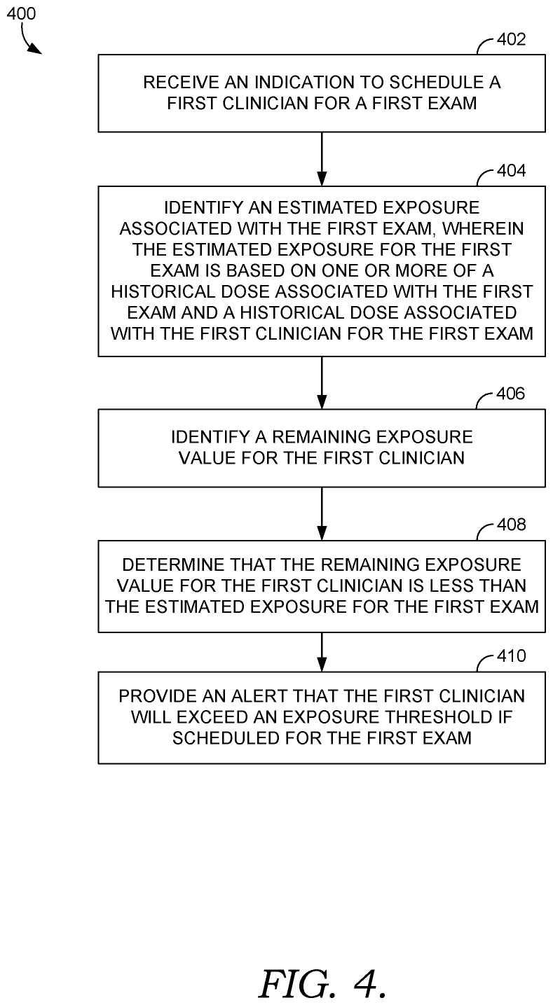

Turning now to FIG. 4, a flow diagram is provided showing a method 400 in accordance with some embodiments of the present invention. Initially, at block 402, an indication is received to schedule a first clinician for a first exam. At block 404, an estimated exposure associated with the first exam is identified, wherein the estimated exposure for the first exam is based on one or more of a historical dose associated with the first exam and a historical dose associated with the first clinician for the first exam. At block 406, a remaining exposure value for the first clinician is identified, wherein the remaining exposure value is an amount of radiation exposure permitted prior to meeting or exceeding an exposure threshold value. At block 408, it is determined that the remaining exposure value for the first clinician is less than the estimated exposure associated with the first exam. At block 410, an alert is generated that the first clinician will exceed the exposure threshold value if scheduled to perform the first exam.

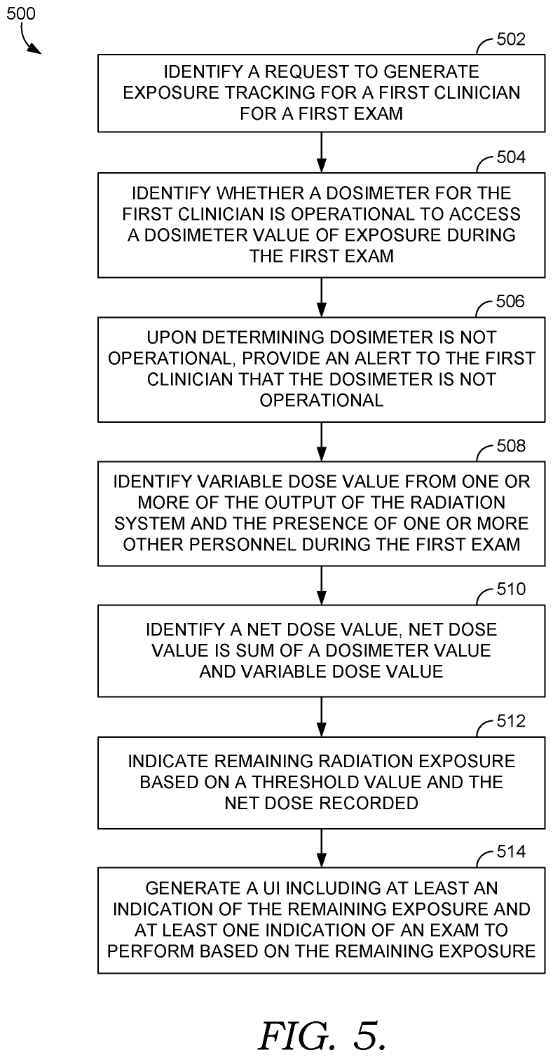

Turning now to FIG. 5, a flow diagram is provided showing a method 500 in accordance with some embodiments of the present invention. Initially, at block 502, a request to generate an exposure tracking graphical user interface (GUI) for a first clinician for a first exam is identified. At block 504, it is identified whether a dosimeter for the first clinician is operational to access a dosimeter exposure value identified for the first exam. At block 506, upon determining that the dosimeter is not operational, an alert is provided to the first clinician that the dosimeter is not operational. At block 508, a variable dose value for the first exam is identified, wherein the variable dose value is identified based on one or more of an output of the radiation system used for the first exam and an exposure value associated with one or more other personnel present during the first exam. At block 510, a net dose value is identified, wherein the net dose value is a sum of the dosimeter value and the variable dose value. At block 512, a remaining exposure value is indicated based on an exposure threshold value and the net dose value. At block 514, the GUI is generated including at least an indication of the remaining exposure value and at least one indication of a second exam the first clinician is eligible to perform based on the remaining exposure value.

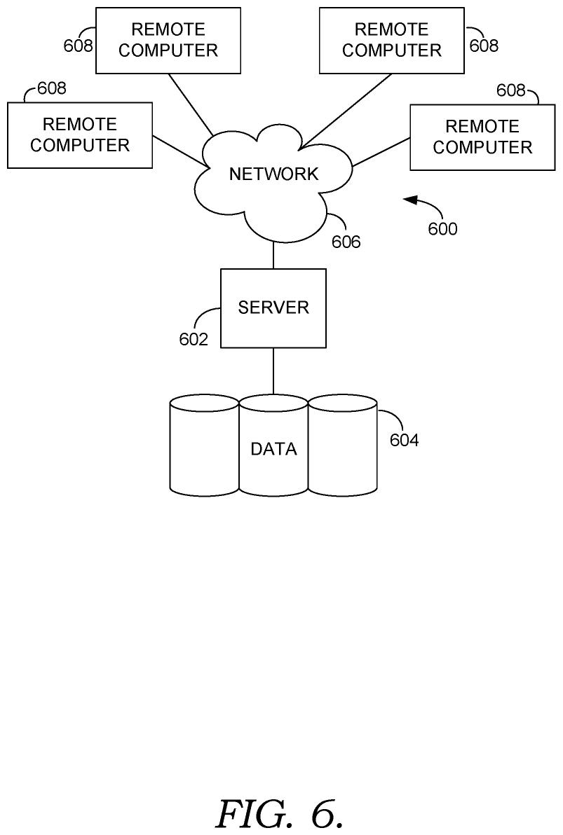

Turning to FIG. 6, it depicts a block diagram of an exemplary environment suitable to implement embodiments of the present invention. The exemplary computing environment 600 is suitable to implement embodiments of the present invention. It will be understood by those of ordinary skill in the art that the exemplary computing environment 600 is just one example of a suitable computing environment and is not intended to limit the scope of use or functionality of the present invention. Similarly, the exemplary computing environment 600 should not be interpreted as imputing any dependency and/or any requirements with regard to each component and combination(s) of components illustrated in FIG. 6. It will be appreciated by those having ordinary skill in the art that the connections illustrated in FIG. 6 are also exemplary as other methods, hardware, software, and devices for establishing a communications link between the components, devices, systems, and entities, as shown in FIG. 6, may be utilized in implementation of the present invention. Although the connections are depicted using one or more solid lines, it will be understood by those having ordinary skill in the art that the exemplary connections of FIG. 6 may be hardwired or wireless, and may use intermediary components that have been omitted or not included in FIG. 6 for simplicity's sake. As such, the absence of components from FIG. 6 should be not be interpreted as limiting the present invention to exclude additional components and combination(s) of components. Moreover, though devices and components are represented in FIG. 6 as singular devices and components, it will be appreciated that some embodiments may include a plurality of the devices and components such that FIG. 6 should not be considered as limiting the number of a devices or components.

Continuing, the exemplary computing environment 600 of FIG. 6 is illustrated as being a distributed environment where components and devices may be remote from one another and may perform separate tasks. The components and devices may communicate with one another and may be linked to each other using a network 606. The network 606 may include wireless and/or physical (e.g., hardwired) connections. Exemplary networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. The network 606, generally, provides the components and devices access to the Internet and web-based applications. The exemplary environment may also be a cloud computing environment.

The exemplary computing environment 600 comprises a computing device in the form of a server 602. Although illustrated as one component in FIG. 6, the present invention may utilize a plurality of local servers and/or remote servers in the exemplary computing environment 600. The server 602 may include components such as a processing unit, internal system memory, and a suitable system bus for coupling to various components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

The server 602 may include or may have access to computer-readable media. Computer-readable media can be any available media that may be accessed by server 602, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 602. Computer storage media does not comprise signals per se.

Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also may be included within the scope of computer-readable media.

In embodiments, the server 602 uses logical connections to communicate with one or more remote computers 608 within the exemplary computing environment 600. In one embodiment, the one or more remote computers 608 comprise external computer systems that leverage object-oriented programming. In embodiments where the network 606 includes a wireless network, the server 602 may employ a modem to establish communications with the Internet, the server 602 may connect to the Internet using Wi-Fi or wireless access points, or the server 602 may use a wireless network adapter to access the Internet. The server 602 engages in two-way communication with any or all of the components and devices illustrated in FIG. 6, using the network 606. Accordingly, the server 602 may send data to and receive data from the remote computers 608 over the network 606.

Although illustrated as a single device, the remote computers 608 may include multiple computing devices. In an embodiment having a distributed network, the remote computers 608 may be located at one or more different geographic locations. In an embodiment where the remote computers 608 are a plurality of computing devices, each of the plurality of computing devices may be located across various locations such as buildings in a campus, medical and research facilities at a medical complex, offices or “branches” of a banking/credit entity, or may be mobile devices that are wearable or carried by personnel, or attached to vehicles or trackable items in a warehouse, for example.

In some embodiments, the remote computers 608 are physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. By way of example, a medical professional may include physicians; medical specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; students; and the like. In other embodiments, the remote computers 608 may be physically located in a non-medical setting, such as a packing and shipping facility or deployed within a fleet of delivery or courier vehicles. Remote computers 608 can also be hosted on a private or public cloud.

Continuing, the exemplary computing environment 600 includes a database 604. In some embodiments, the database 604 and at least the server 602, together, form a relational database management system. Although shown as a single component, the database 604 may be implemented using multiple data stores that are communicatively coupled to one another, independent of the geographic or physical location of a memory device. Exemplary data stores may also store data in the form of electronic records, for example, electronic medical records of patients, transaction records, billing records, task and workflow records, chronological event records, and the like. Database 604 can also be hosted on a private or public cloud.

Generally, the database 604 includes physical memory that is configured to store information encoded in data. For example, the database 604 may provide storage for computer-readable instructions, computer-executable instructions, data structures, data arrays, computer programs, applications, and other data that supports the functions and action to be undertaken using the exemplary computing environment 600 and components shown in exemplary FIG. 6.

In a computing environment having distributed components that are communicatively coupled via the network 606, program modules may be located in local and/or remote computer storage media including, for example only, memory storage devices. Embodiments of the present invention may be described in the context of computer-executable instructions, such as program modules, being executed by a computing device. Program modules may include, but are not limited to, routines, programs, objects, components, and data structures that perform particular tasks or implement particular data types. In embodiments, the server 602 may access, retrieve, communicate, receive, and update information stored in the database 604, including program modules. Accordingly, the server 602 may execute, using a processor, computer instructions stored in the database 604 in order to perform embodiments described herein.

Although internal components of the devices in FIG. 6, such as the server 602, are not illustrated, those of ordinary skill in the art will appreciate that internal components and their interconnection are present in the devices of FIG. 6. Accordingly, additional details concerning the internal construction of the device are not further disclosed herein.

The present invention has been described in relation to particular embodiments, which are intended in all respects to be illustrative rather than restrictive. Further, the present invention is not limited to these embodiments, but variations and modifications may be made without departing from the scope of the present invention.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations.

What is claimed is:

1. One or more non-transitory computer-readable media having executable instructions embodied thereon that, when executed by a processor of a computer device, perform a method, the method comprising:
receiving an indication to schedule a first clinician for a first exam;
identifying an estimated exposure associated with the first exam, wherein the estimated exposure for the first exam is based on one or more of a historical dose associated with the first exam and a historical dose associated with the first clinician for the first exam;
identifying a remaining exposure value for the first clinician, wherein the remaining exposure value is an amount of radiation exposure permitted prior to meeting or exceeding an exposure threshold value;
determining that the remaining exposure value for the first clinician is less than the estimated exposure associated with the first exam; and
generating an alert that the first clinician will exceed the exposure threshold value if scheduled to perform the first exam.

2. The media of claim 1, wherein the method further comprises:
identifying a clinician critical indicator for the first clinician;
identifying an exam critical indicator for the first exam; and
determining that the clinician critical indicator and the exam critical indicator are a match.

3. The media of claim 2, wherein the clinician critical indicator and the exam critical indicator are both critical.

4. The media of claim 2, wherein the clinician critical indicator and the exam critical indicator are both non-critical.

5. The media of claim 1, wherein the method further comprises:
based on determining that the remaining exposure value for the first clinician is less than the estimated exposure associated with the first exam, identifying one or more backup clinicians to schedule for the first exam, wherein the one or more backup clinicians are identified based on their remaining exposure values being at least equal to the estimated exposure associated with the first exam.

6. The media of claim 5, wherein the one or more backup clinicians are provided by filtering one or more other clinicians that are associated with remaining exposure values less than the estimated exposure associated with the first exam.

7. The media of claim 1, wherein the remaining exposure value for the first clinician is calculated based on a difference between the exposure threshold value and an amount of exposure already recorded prior to the first exam.

8. One or more non-transitory computer-readable media having executable instructions embodied thereon that, when executed by a processor of a computer device, perform a method, the method comprising:
identifying a request to generate an exposure tracking graphical user interface (GUI) for a first clinician for a first exam;
identifying whether a dosimeter for the first clinician is operational to access a dosimeter exposure value identified for the first exam;
upon determining that the dosimeter is not operational, providing an alert to the first clinician that the dosimeter is not operational;
identifying a variable dose value for the first exam, wherein the variable dose value is identified based on one or more of an output of the radiation system used for the first exam and an exposure value associated with one or more other personnel present during the first exam;
identifying a net dose value, wherein the net dose value is a sum of the dosimeter value and the variable dose value;
indicating a remaining exposure value based on an exposure threshold value and the net dose value; and
generating the GUI including at least an indication of the remaining exposure value and at least one indication of a second exam the first clinician is eligible to perform based on the remaining exposure value.

9. The media of claim 8, wherein the variable dose value is further identified based on a patient exposure value of a patient during the first exam.

10. The media of claim 9, wherein the variable dose value is an average of the exposure value associated with one or more other personnel present during the first exam.

11. The media of claim 8, wherein the method further comprises identifying an exposure value of the second exam the first clinician is eligible to perform.

12. The media of claim 11, wherein the exposure value of the second exam the first clinician is eligible to perform is based on a historical exposure value for the second exam.

13. The media of claim 11, wherein the exposure value of the second exam the first clinician is eligible to perform is based on a historical exposure value of the first clinician for the second exam.

14. The media of claim 8, wherein the GUI further comprises each of a critical indicator and a non-critical indicator, wherein critical indicators and non-critical indicators are associated with one or more of a clinician or an exam.

15. The media of claim 14, wherein the method further comprises identifying a critical indicator for the first clinician and a critical indicator for the second exam.

16. The media of claim 14, wherein the method further comprises determining that the critical indicator for the first clinician and the critical indicator for the second exam are a match.

17. A system for intelligent radiation dose exposure monitoring, the system comprising:
one or more processors configured to:
receive an indication to schedule a first clinician for a first exam;
identify an estimated exposure associated with the first exam, wherein the estimated exposure for the first exam is based on one or more of a historical dose associated with the first exam and a historical dose associated with the first clinician for the first exam;
identify a remaining exposure value for the first clinician, wherein the remaining exposure value is an amount of radiation exposure permitted prior to meeting or exceeding an exposure threshold value;
determine that the remaining exposure value for the first clinician is less than the estimated exposure associated with the first exam; and
generate an alert that the first clinician will exceed the exposure threshold value if scheduled to perform the first exam.

18. The system of claim 17, wherein the one or more processors is further configured to:
based on determining that the remaining exposure value for the first clinician is less than the estimated exposure associated with the first exam, identify one or more backup clinicians to schedule for the first exam, wherein the one or more backup clinicians are identified based on their remaining exposure values being at least equal to the estimated exposure associated with the first exam.

19. The system of claim 18, wherein the one or more backup clinicians are provided by filtering one or more other clinicians that are associated with remaining exposure values less than the estimated exposure associated with the first exam.

20. The system of claim 17, wherein the remaining exposure value for the first clinician is calculated based on a difference between the exposure threshold value and an amount of exposure already recorded prior to the first exam.
